(19) United States
(12) Patent Application Publication
(10) Pub. No.: US 2008/0091005 A1
(43) Pub. Date: Apr. 17, 2008

(54) MODIFIED NUCLEOTIDES, METHODS FOR MAKING AND USING SAME

(75) Inventors: Hongyi Wang, Houston, TX (US); Xiaolian Gao, Houston, TX (US); Peilin Yu, Houston, TX (US); Mitsu Reddy, Pearland, TX (US); Susan H. Hardin, College Station, TX (US); Tommie Lincecum, Houston, TX (US); Amy Williams, Houston, TX (US); Norha Deluge, Houston, TX (US); Yuri Belosludtsev, The weedlands, TX (US); Steven M. Menchen, Fremont, CA (US); Joe Y.L. Lam, Castro Valley, CA (US); Jer-Kang Chen, Palo Alto, CA (US)

Correspondence Address: ROBERT W STROZIER, P.L.L.C, PO BOX 429, BELLAIRE, TX, 77402-0429, US

(73) Assignees: VISIGEN BIOTECHNOLOGIES, INC., Houston, TX (US); APPLIED BIOSYSTEMS, Foster City, CA (US)

(21) Appl. No.: 11/781,160

(22) Filed: Jul. 20, 2007

Related U.S. Application Data
(60) Provisional application No. 60/832,097, filed on Jul. 20, 2006.

(51) Int. Cl. C07H 21/04

(52) U.S. Cl. 536/26.6

(57) ABSTRACT

Modified nucleotides are disclosed for use in single molecule sequencing, methods for making the modified nucleotides and method for using the modified nucleotides. Linkers for making the modified nucleotide are also disclosed.

### RELATED APPLICATIONS

This application claims provisional priority to U.S. Provisional Patent Application No. 60/832,097 filed Jul. 20, 2006 (20 Jul. 2006).

### BACKGROUND OF THE INVENTION

1. Field of the Invention

The present invention relates to modified nucleotides and methods for making and using same.

More particularly, the present invention relates to modified nucleotides including a natural or synthetic nucleotide having bonded to at least one site of the nucleotide a linker. The invention also relates to a modified nucleotide including a natural or synthetic nucleotide having bonded to at least one site a linker including at least one detectable group or moiety bonded to at one site of the linker. The invention also relates to method for making and using same.

2. Description of the Related Art

As single molecule sequencing advance ever closing to the ultimate goal of obtaining sequencing information from one or a large number of single molecule active sequencing sites in a field of view of a real time or near real time detection system, the need for modified nucleotide capable of detecting in such systems advance as well.

Although many modified nucleotides have been devised, there is a need in the art for modified nucleotides having a detectable group bonded thereto for use in such single molecule sequencing systems.

### SUMMARY OF THE INVENTION

General Structures

The present invention provides modified nucleotides of the general formula (I):
DG-E′-G-E-Nu (I)
where:

DG is a detectable group,

E and E′ are the same and different group including a central main group element selected from the group consisting of boron (B), carbon (C), nitrogen (N), oxygen (O), silicon (Si), phosphorus (P), sulfur (S), gallium (Ga) and germanium (Ge),

G is a linking group, and

Nu is a natural or synthetic nucleotide.

G can include a linear or branched alkenyl group or an alkenyl group including a central ring structure.

Structures with Ring Structure in the Core

The present invention provides modified nucleotides of the general formula (II):
DG-E′-R²-A—R¹-E-Nu (II)
where:

DG is a detectable group,

E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),

R¹ and R² are the same or different and are carbenzyl groups,

A is a ring structure, and

Nu is a natural or synthetic nucleotide.

The present invention also provides modified nucleotides of the general formulas (III or IIIa) (γ-phosphate modified):
DG-E′-R²-A-R¹-E-P(O₂)OP(O₂)OP(O₂)-Sugar-Base (III)
DG-E′-R²-A-R¹-E-P(O₂)OP(OZ¹)OP(OZ²)-Sugar-Base (IIIa)
where:

DG is a detectable group,

E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),

R¹ and R² are the same or different and are is carbenzyl groups,

A is a ring structure,

Sugar is a sugar moiety,

Base is a natural or synthetic nucleotide base and

Z¹ or Z² are the same or different and are groups that either modify incorporation timing or enhancing detection of the detectable group as described herein.

The present invention also provides modified nucleotides of the general formulas (IV or IVa) (β-phosphate modified):
DG-E′-R²-A-R¹-E-P(O)(OP(O₂)OH)OP(O₂)-Sugar-Base (IV)
DG-E′-R²-A-R¹-E-P(O)(OP(OZ¹)OH)OP(OZ²)-Sugar-Base (IVa)
where:

DG is a detectable group,

E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),

R¹ and R² are the same or different and are is carbenzyl groups,

A is a ring structure,

Sugar is a sugar moiety,

Base is a natural or synthetic nucleotide base and

Z¹ or Z² are the same or different and are groups that either modify incorporation timing or enhancing detection of the detectable group as described herein.

The present invention also provides modified nucleotides of the general formula (V) (α-phosphate modified):
DG-E′-R²-A-R¹-E-P(O)(OP(O₂)OP(O₂)OH)-Sugar-Base (V)
where:

DG is a detectable group,

E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),

R¹ and R² are the same or different and are is carbenzyl groups,

A is a ring structure,

Sugar is a sugar moiety, and

Base is a natural or synthetic nucleotide base.

The present invention also provides modified nucleotides of the general formula (VI) (sugar modified):
DG-E′-R²-A-R¹-E-Sugar(P(O₂)OP(O₂)OP(O₂)OH)Base (VI)
where:

DG is a detectable group,

E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),

R¹ and R² are the same or different and are is carbenzyl groups,

A is a ring structure,

Sugar is a sugar moiety, and

Base is a natural or synthetic nucleotide base.

The present invention also provides modified nucleotides of the general formula (VII) (base modified):
DG-E′-R²-A-R¹-E-Base-Sugar-P(O₂)OP(O₂)OP(O₂)OH (VII)
where:

DG is a detectable group,

E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),

R¹ and R² are the same or different and are is carbenzyl groups,

A is a ring structure,

Sugar is a sugar moiety, and

Base is a natural or synthetic nucleotide base.

In formulas (II-VII), the ring structure A can be saturated, unsaturated or aromatic or can include a mixture of saturated, unsaturated, or aromatic rings. Each ring in a ring structure include from 3 to about 12 main group elements. Of course, higher ordered rings are also included. Each carbyl group and each carbenzyl group include from 1 to 40 carbon, where one or more of the carbon atoms can be replaced with a hetero atoms selected from the group consisting of B, C, Si, Ge, N, P, As, O, S, or Se and having sufficient hydrogen atoms to satisfy the valency of the group, where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions. It should be recognized that the linker group comprises —R²-A-R¹— in the formulas (II-VII)

The present invention also provides a method for using the compounds of Formulas (II-VII) in single molecule sequencing including the step adding a compound of Formulas (II-VII) and detecting the detectable group before, during and/or after incorporation of one or a series of compounds of Formulas (II-VII).

The present invention also provides a method for using the compounds of Formulas (II-VII) in single molecule sequencing including the step adding a compound of Formulas (II-VII), where the detectable group is a fluorophore and detecting light from the fluorophore before, during and/or after incorporation of one or a series of compounds of Formulas (II-VII).

The present invention also provides a method for using the compounds of Formulas (II-VII) in single molecule sequencing including the step adding a compound of Formulas (II-VII), where the detectable group is an acceptor fluorophore and detecting light from the acceptor fluorophore after fluorescence resonance energy transfer from a donor fluorophore before, during and/or after incorporation of one or a series of compounds of Formulas (II-VII).

The Formulas (II-VII) can also includes other groups at different location of the nucleotide including the phosphates, sugar and/or base. The additional groups are not intended to be detectable groups, but are groups designed to change the incorporation timing of the nucleotide modified with these additional groups. The additional groups can be atom replacements on the phosphates such as replacing an oxygen atom with a sulfur, nitrogen containing group, a carbon containing group, a boron containing group or any other group or atom that will change the incorporation timing of the nucleotide. In this way, sequencing can be performed with fewer distinct detectable groups, e.g., dATP and dTTP could be have the same detectable group, but modified with different additional groups so that one incorporates much faster than the other so that the detection signature of the incorporation will be distinguishable. These additional groups could also improve detectability of the detectable group by interacting with detectable group in a way that changes during the incorporation cycle—binding, incorporation, and pyrophosphate release.

Structures with Chains in the Core

The present invention provides modified nucleotides of the general formula (VIII):
DG-E′-R-E-Nu (VIII)
where:

DG is a detectable group,

E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 2 and 10), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),

R is a carbenzyl group, and

Nu is a natural or synthetic nucleotide.

The present invention also provides modified nucleotides of the general formulas (IX or IXa) (γ-phosphate):
DG-E′-R-E-P(O₂)OP(O₂)OP(O₂)-Sugar-Base (IX)
DG-E′-R-E-P(O₂)OP(OZ¹)OP(OZ²)-Sugar-Base (IXa)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety,
Base is a natural or synthetic nucleotide base, and
Z¹ or Z² are the same or different and are groups that either modify incorporation timing or enhancing detection of the detectable group as described herein.

The present invention also provides modified nucleotides of the general formulas (X or Xa) (β-phosphate):
DG-E′-R-E-P(O)(OP(O₂)OH)OP(O₂)-Sugar-Base (X)
DG-E′-R-E-P(O)(OP(OZ¹)OH)OP(OZ²)-Sugar-Base (Xa)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety,
Base is a natural or synthetic nucleotide base, and
Z¹ or Z² are the same or different and are groups that either modify incorporation timing or enhancing detection of the detectable group as described herein.

The present invention also provides modified nucleotides of the general formula (XI) (α-phosphate):
DG-E′-R-E-P(O)(OP(O₂)OP(O₂)OH)-Sugar-Base (XI)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety, and
Base is a natural or synthetic nucleotide base.

The present invention also provides modified nucleotides of the general formula (XII):
DG-E′-R-E-Sugar(P(O₂)OP(O₂)OP(O₂)OH)Base (XII)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety, and
Base is a natural or synthetic nucleotide base.

The present invention also provides modified nucleotides of the general formula (XIII):
DG-E′-R-E-Base-Sugar-P(O₂)OP(O₂)OP(O₂)OH (XIII)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety, and
Base is a natural or synthetic nucleotide base.

Each carbyl group and each carbenzyl group include from 1 to 40 carbon, where one or more of the carbon atoms can be replaced with a hetero atoms selected from the group consisting of B, C, Si, Ge, N, P, As, O, S, or Se and having sufficient hydrogen atoms to satisfy the valency of the group, where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions. It should be recognized that the linker comprises —R— in formulas (VIII-XIII).

The present invention also provides a method for using the compounds of Formulas (VIII-XIII) in single molecule sequencing including the step adding a compound of Formulas (VIII-XIII) and detecting the detectable group before, during and/or after incorporation of one or a series of compounds of Formulas (VIII-XIII).

The present invention also provides a method for using the compounds of Formulas (VIII-XIII) in single molecule sequencing including the step adding a compound of Formulas (VIII-XIII), where the detectable group is a fluorophore and detecting light from the fluorophore before, during and/or after incorporation of one or a series of compounds of Formulas (VIII-XIII).

The present invention also provides a method for using the compounds of Formulas (VIII-XIII) in single molecule sequencing including the step adding a compound of Formulas (VIII-XIII), where the detectable group is an acceptor fluorophore and detecting light from the acceptor fluorophore after fluorescence resonance energy transfer from a donor fluorophore before, during and/or after incorporation of one or a series of compounds of Formulas (VIII-XIII).

The Formulas (VIII-XIII) can also includes other groups at different location of the nucleotide including the phosphates, sugar and/or base. The additional groups are not intended to be detectable groups, but are groups designed to change the incorporation timing of the nucleotide modified with these additional groups. The additional groups can be atom replacements on the phosphates such as replacing an oxygen atom with a sulfur, nitrogen containing group, a carbon containing group, a boron containing group or any other group or atom that will change the incorporation timing of the nucleotide. In this way, sequencing can be performed with fewer distinct detectable groups, e.g., dATP and dTTP could be have the same detectable group, but modified with different additional groups so that one incorporates much faster than the other so that the detection signature of the incorporation will be distinguishable. These additional groups could also improve detectability of the detectable group by interacting with detectable group in a way that changes during the incorporation cycle—binding, incorporation, and pyrophosphate release.

### BRIEF DESCRIPTION OF THE DRAWINGS

The invention can be better understood with reference to the following detailed description together with the appended illustrative drawings in which like elements are numbered the same.

FIG. 1 depicts exemplary single ring linker structures of this invention.

FIG. 2 depicts other exemplary single ring linker structures of this invention.

FIGS. 3&4 depicts exemplary binary ring linker structures of this invention.

FIG. 5 depicts exemplary trinary ring linker structures of this invention.

FIG. 6 depicts exemplary dyes for use in the modified nucleotide structures of this invention.

FIG. 7 depicts two synthetic schemes for preparing modified nucleotide triphosphates, where the modification is a linker terminating in a dye.

Figure depicts a synthetic scheme for preparing modified nucleotide triphosphates, where the modification is a linker terminating in a dye.

FIG. 9 depicts a synthetic scheme for preparing modified nucleotide triphosphates, where the modification is a linker terminating in a dye.

FIG. 10 depicts a synthetic scheme for preparing modified nucleotide triphosphates, where the modification is a linker terminating in a dye.

FIG. 11 depicts a synthetic scheme for preparing modified nucleotide triphosphates, where the modification is a linker terminating in a dye.

FIG. 12 depicts a synthetic scheme for preparing modified nucleotide triphosphates, where the modification is a linker terminating in a dye.

FIG. 13 depicts a synthetic scheme for preparing modified nucleotide triphosphates, where the modification is a linker terminating in a dye.

### DETAILED DESCRIPTION OF THE INVENTION

The inventors have found that modified nucleotide for use in sequencing experiments can be constructed from a linker group including a central group and terminal groups including a main group element. The central group can be a linear carbenzyl group, a branched carbenzyl group or a arenyl group. The hydroxy group is adapted to react with a nucleotide at a phosphate moiety, a sugar moiety and/or base moiety. The nucleotide can be naturally occurring or human created, where the human created nucleotide have altered incorporation rates and/or fidelities. The amino group is adapted to react with a detectable groups such as a fluorescent dye.

The present invention broadly relates to modified nucleotides of the general formula (I):
DG-E′-R²-G-R¹-E-Nu (I)
where DG is a detectable group, E and E′ are the same and different group including a central main group element, R¹ and R² are the same or different and are carbenzyl groups, G is a central group, and Nu is a natural or synthetic nucleotide. The central group G can be a ring structure or an alkenyl group. If it is an alkenyl group, then R²-G-R¹ can be re-designated by the symbol R.

The present invention relates also broadly to modified nucleotide including a linker having a central ring structure, an amino terminated moiety and a hydroxy terminated moiety. The central ring structure can be a saturated ring structure, a partially unsaturated ring structure or an aromatic ring structure. The modified nucleotides including compounds of the general formula (II):
DG-E′-R²-A —R¹-E-Nu (II)
where DG is a detectable group, E and E′ are the same and different and are a carbon group (C(H)₂, C(HR³) or C(R³)₂), an oxygen atom (O), a sulfur atom (S), an amino group (N(R³)), an phosphano group (P(R³)), phosphito (P(OR³)O), phosphate (P(O₂)O), polyphosphate (P(O₂)O)ₙ (n is an integer having a value between 3 and 12), silyl (Si(R³)₂), siloxyl (Si(OR³)₂) carboxy group (C(O)O), keto (C(O)), amido group (C(O)N(R³)), urea group (N(R³)C(O)N(R³)), carbonate (OC(O)O), urethane group (OC(O)N(R³), is a nitrogen atom, R³ is a hydrogen atom, a carbyl group or is absent depending on the structure (e.g., E′ or E is a nitrogen atom doubly bonded to DG or to R²), R¹ and R² are the same or different and are is carbenzyl groups, A is a ring structure, and Nu is a natural or synthetic nucleotide. The ring structure A is saturated, unsaturated or aromatic or can include a mixture of saturated, unsaturated, or aromatic rings. Each carbyl group and each carbenzyl group include from about 1 to about 40 carbon atoms, where one or more of the carbon atoms is replaced with an hetero atom or an hetero atom containing group.

The present invention relates also broadly to modified nucleotides of the general formula (III):
DG-E′-R-E-Nu (VIII)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group, and
Nu is a natural or synthetic nucleotide.
   Methods for Preparing Modified Nucleotides

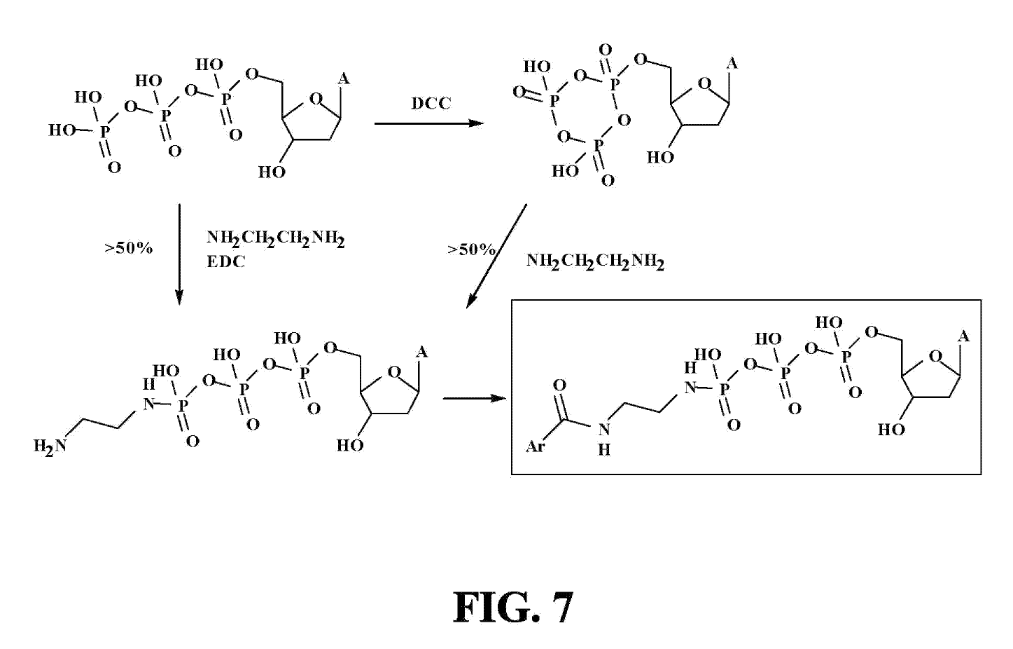

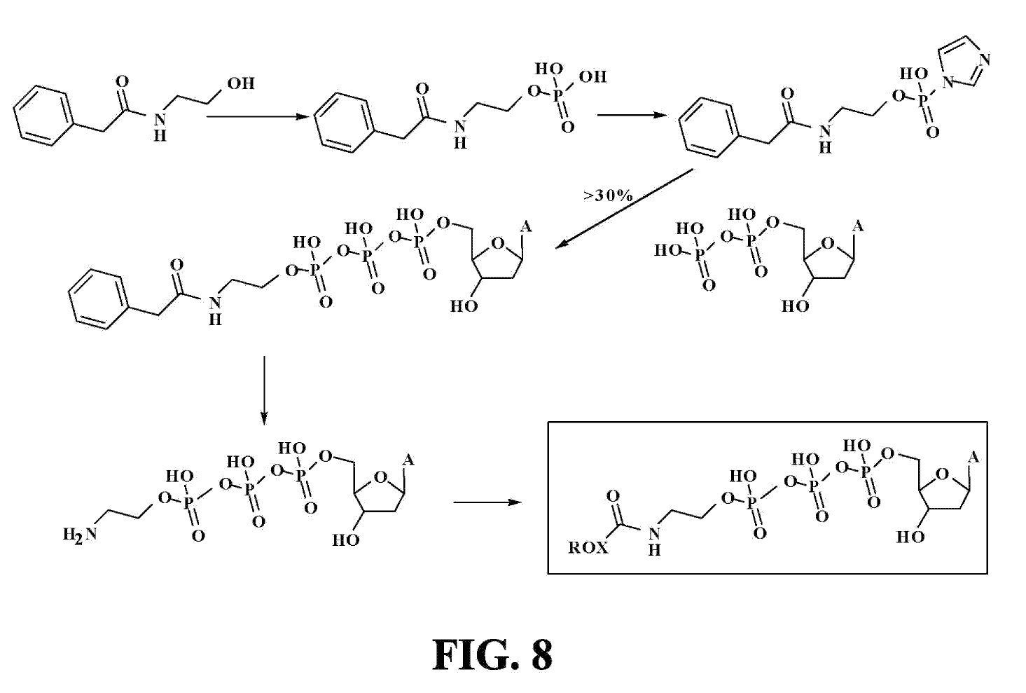

The present invention also relates to methods for preparing modified nucleotides, especially gamma (γ) phosphate modified nucleotides. One such method includes the step of reacting a nucleotide triphosphate with a diamine in N-ethyl-N′-(3-dimethylaminopropyl)carbodiimide (EDC) or pre-cyclizing a nucleotide triphosphate in N,N-dicyclohexylcarbodiimide (DCC) and then reacted with a diamine. Both routes produce a diamine functionalized gamma (γ) phosphate modified nucleotide terminating in a free amino group in yields greater than about 50%. The free amino group can then be treated with an acid, an anhydride or an acid chloride to produce an amide functionalized gamma (γ) phosphate modified nucleotide, where the amido group (which can bear a fluorophore) is separated from the gamma (γ) phosphate by a linker or linking group the portion of the diamine excluding the two terminal amino groups —H₂N-L-NH₂, where L is the linking group which can be a R²-G-R¹ motif, R²-A-R¹ motif or an R motif as shown in Formulas (I), (II), and (VIII) above. These two methods are shown in pictorially in FIG. 7.

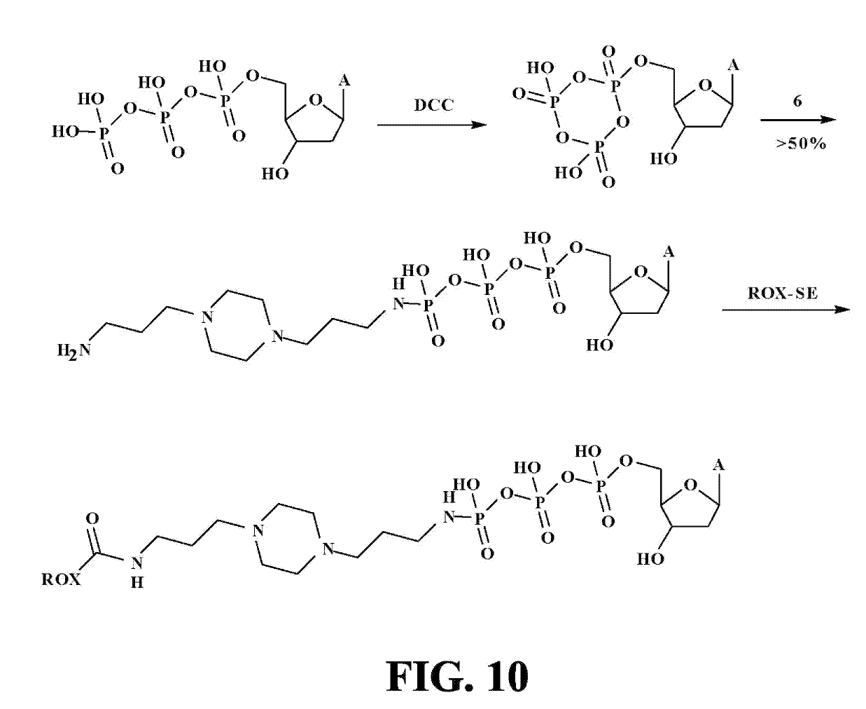

The second method set forth above can also be used to prepare modified gamma (γ) phosphate nucleotide triphosphates, where the linker molecule is of the general motif E′-R²-A-R¹-E as set forth in Formula (II) and shown pictorially in FIG. 10.

Another such method includes the step of reacting a linker molecule including an N-protected, terminal amino group and a hydroxy terminal group (Protector —HN-L-OH) with phosphate to produce a linker molecule bearing a terminal phosphate group (Protector —HN-L-OP(O)OH₂). Next, the terminal phosphate linker molecule is activated with carbonyldiimidazole to produce an imidazole activated terminal phosphate linker molecule. The imidazole activated terminal phosphate linker molecule is reacted with a nucleotide diphosphate to produce a protected-amino terminated, functionalized gamma (γ) phosphate modified nucleotide triphosphate. The protected-amino terminated, functionalized gamma (γ) phosphate modified nucleotide triphosphate is then deprotected and the free amine is then treated with an acid, an anhydride or an acid chloride to produce an amide functionalized gamma (γ) phosphate modified nucleotide, where the amido group (which can bear a fluorophore) is separated from the gamma (γ) phosphate by a linker or linking group the portion of the diamine excluding the two terminal amino groups —H₂N-L-OH, where L is the linking group which can be a R²-G-R¹ motif R²-A-R¹ motif or an R motif as shown in Formulas (I), (II), and (VIII) above. This method is shown in pictorially in FIG. 8.

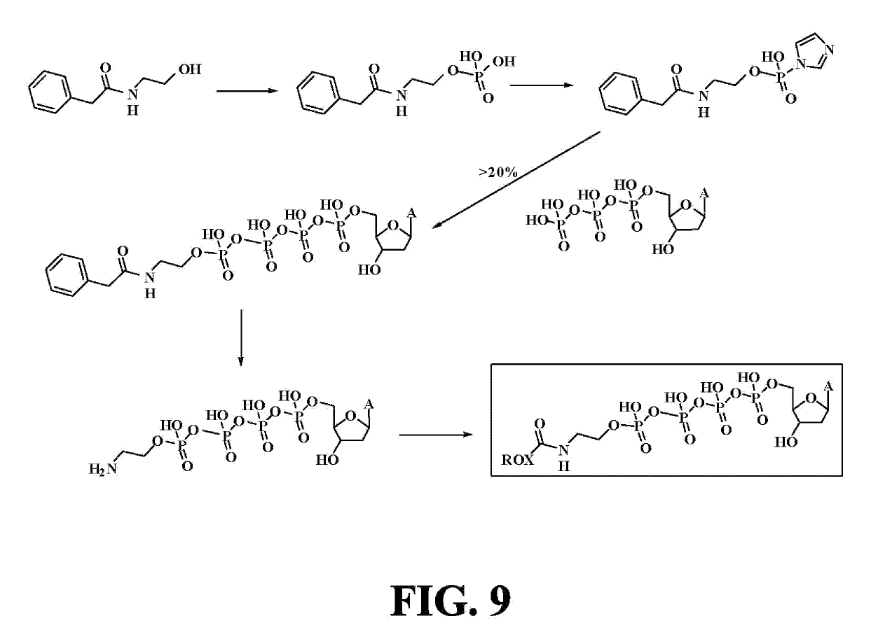

The above multi-step reaction can also be used to produce functionalized nucleotide polyphosphates. This method is shown in pictorially in FIG. 9, which evidences a general synthesis for functionalized nucleotide tetra phosphates.

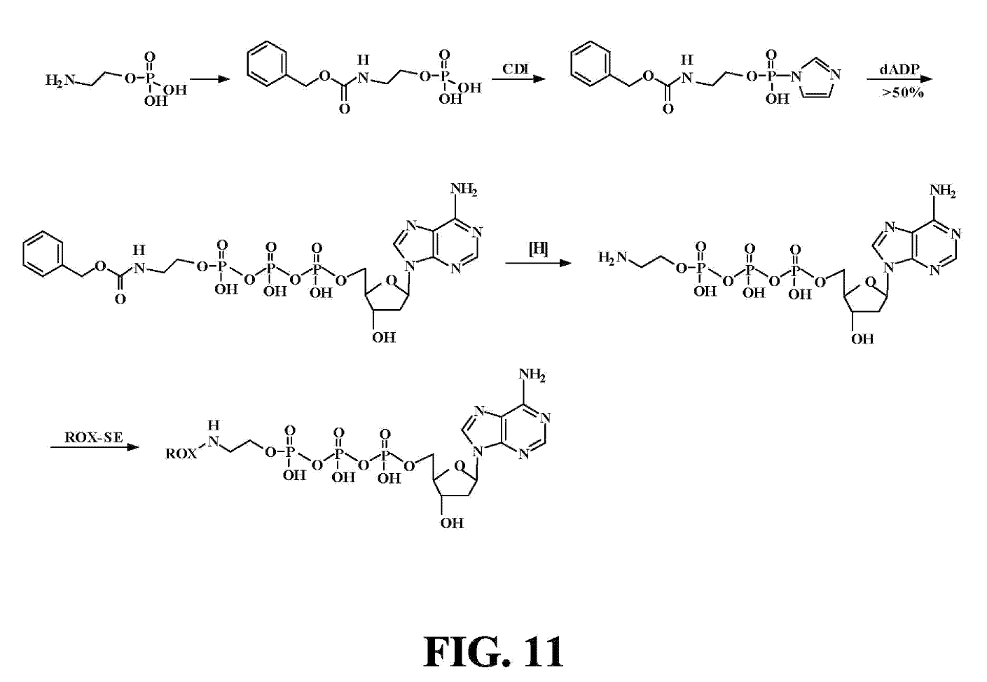

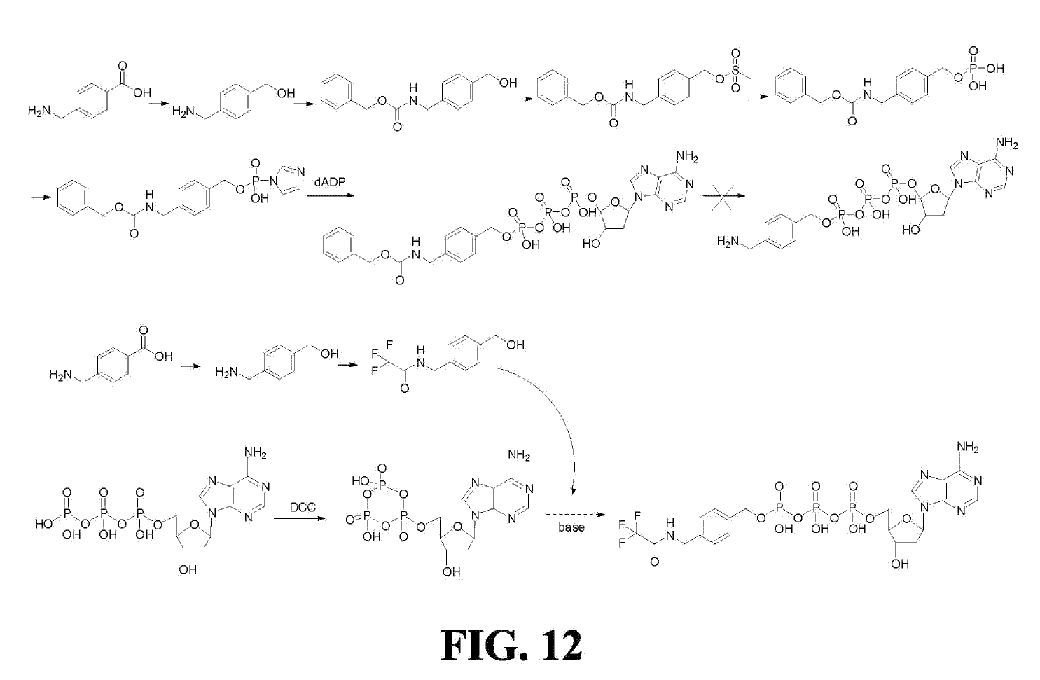

An alternate multi-step reaction similar to the multi-step reaction above can also be used to produce functionalized nucleotide polyphosphates. The alternate reaction starts with an amino and phosphate terminated linker molecule, where the amino group is then protected before reacting the phosphate linker with carbonyldiimidazole. This method is shown in pictorially in FIG. 11, which evidences a general synthesis for functionalized nucleotide tetra phosphates.

Another such method includes the step of reducing an amine terminated alkylated benzoic acid to produce an amine terminated alkylated, a hydroxy terminated alkylate benzene linker molecule. The linker is then amine protected and the hydroxy group is sulfonated. The sulfonated, protected linker molecule is then reacted with phosphate to form a phosphate, protected linker molecule. The phosphate, protected linker molecule is then activated with imidazole and reacted with a nucleotide diphosphate to form a gamma phosphate functionalized nucleotide triphosphate. However, deprotecting of the amino group resulted in very poor yields. Thus, this method is of little utility in forming gamma phosphate functionalized nucleotide triphosphate. However, an alternate reaction scheme did result in a general synthetic scheme to prepare gamma phosphate functionalized nucleotide triphosphates. The alternate synthesis includes reducing an amine terminated alkylated benzoic acid to produce an amine terminated alkylated, a hydroxy terminated alkylate benzene linker molecule. The linker is then amine protected with TFA protecting group. The TFA protected linker molecule is then reacted with a cyclized nucleotide triphosphate to produce a TFA protected gamma phosphate functionalized nucleotide triphosphate. Deprotecting and dye treatment produces dye gamma phosphate functionalized nucleotide triphosphates.

For additional information on DNA sequencing, data acquisition and analysis, monomers, monomers synthesis, or other features of system that are amenable to detection using the apparatuses and methods of this invention, the reader is referred to United States patent, Published patent application and Pending patent application Ser. Nos. 09/901,782; 10/007,621; 11/007,794; 11/671,956; 11/694,605; 2006-0078937; U.S. Pat. No. 6,982,146; U.S. Pat. No. 7,169,560; U.S. Pat. No. 7,220,549, 20070070349; 20070031875; 20070012113; 20060286566; 20060252077; 20060147942; 200601336144; 20060024711; 20060024678; 20060012793; 20060012784; 20050100932; incorporated herein by reference.

Suitable Reagents

Suitable detectable agents include, without limitation, any group that is detectable by a known or yet to be invented analytical technique. Exemplary examples include, without limitation, fluorophores or chromophorers, group including one or a plurality of nmr active atoms (²H, ¹¹B, ¹³C, ¹⁵N, ¹⁷O, ¹⁹F, ²⁷Al, ²⁹Si, ³¹P, nmr active transition metals, nmr active actinide metals, nmr active lanthanide metals), IR active groups, nearIR active groups, Raman active groups, UV active groups, X-ray active groups, light emitting quantum dots, light emitting nano-structures, or other structures or groups capable of direct detection or that can be rendered detectable or mixtures or combinations thereof.

Suitable atomic tag for use in this invention include, without limitation, any atomic element amenable to attachment to a specific site in a polymerizing agent or dNTP, especially Europium shift agents, nmr active atoms or the like.

Suitable atomic tag for use in this invention include, without limitation, any atomic element amenable to attachment to a specific site in a polymerizing agent or dNTP, especially fluorescent dyes such as d-Rhodamine acceptor dyes including dichloro[R110], dichloro[R6G], dichloro[TAMRA], dichloro[ROX] or the like, fluorescein donor dye including fluorescein, 6-FAM, or the like; Acridine including Acridine orange, Acridine yellow, Proflavin, or the like; Aromatic Hydrocarbon including 2-Methylbenzoxazole, Ethyl p-dimethylaminobenzoate, Phenol, benzene, toluene, or the like; Arylmethine Dyes including Auramine O, Crystal violet, Crystal violet, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dye including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, Thiacarbocyanine (C3) dye, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), 4-(dicyanomethylene)-2-methyl-6-(p-dimethylaminostyryl)-4H-pyran (DCM), 4-Dimethylamino-4′-nitrostilbene, Merocyanine 540, or the like; Miscellaneous Dye including 4′,6-Diamidino-2-phenylindole (DAPI), 4′,6-Diamidino-2-phenylindole (DAPI), 7-Benzylamino-4-nitrobenz-2-oxa-1,3-diazole, Dansyl glycine, Dansyl glycine, Hoechst 33258, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, Nile Red, Nile blue, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridyl)ruthenium(II), Tetrapyrrole, Bilirubin, Chlorophyll a, Chlorophyll b, Diprotonated-tetraphenylporphyrin, Hematin, Magnesium octaethylporphyrin, Magnesium octaethylporphyrin (MgOEP), Magnesium phthalocyanine (MgPc), Magnesium phthalocyanine (MgPc), Magnesium tetramesitylporphyrin (MgTMP), Magnesium tetraphenylporphyrin (MgTPP), Octaethylporphyrin, Phthalocyanine (Pc), Porphin, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), Zinc tetramesitylporphyrin (ZnTMP), Zinc tetramesitylporphyrin radical cation, Zinc tetraphenylporphyrin (ZnTPP), or the like; Cy3, Cy3B, Cy5, Cy5.5, Atto590, Atto610, Atto611, Atto611x, Atto620, Atto655, Alexa488, Alexa546, Alexa594, Alexa610, Alexa610x, Alexa633, Alexa647, Alexa660, Alexa680, Alexa700, Bodipy630, DY610, DY615, DY630, DY632, DY634, DY647, DY680, DyLight647, HiLyte647, HiLyte680, LightCycler (LC) 640, Oyster650, ROX, TMR, TMR5, TMR6; Xanthenes including Eosin Y, Fluorescein, Fluorescein, Rhodamine 123, Rhodamine 6G, Rhodamine B, Rose bengal, Sulforhodamine 101, or the like; or mixtures or combination thereof or synthetic derivatives thereof or FRET fluorophore-quencher pairs including DLO-FB1 (5′-FAM/3′-BHQ-1) DLO-TEB1 (5′-TET/3′-BHQ-1), DLO-JB1 (5′-JOE/3′-BHQ-1), DLO-HB1 (5′-HEX/3′-BHQ-1), DLO-C3B2 (5′-Cy3/3′-BHQ-2), DLO-TAB2 (5′-TAMRA/3′-BHQ-2), DLO-RB2 (5′-ROX/3′-BHQ-2), DLO-C5B3 (5′-Cy5/3′-BHQ-3), DLO-C55B3 (5′-Cy5.5/3′-BHQ-3), MBO-FB1 (5′-FAM/3′-BHQ-1), MBO-TEB1 (5′-TET/3′-BHQ-1), MBO-JB1 (5′-JOE/3′-BHQ-1), MBO-HB1 (5′-HEX/3′-BHQ-1), MBO-C3B2 (5′-Cy3/3′-BHQ-2), MBO-TAB2 (5′-TAMRA/3′-BHQ-2), MBO-RB2 (5′-ROX/3′-BHQ-2); MBO-C5B3 (5′-Cy5/3′-BHQ-3), MBO-C55B3 (5′-Cy5.5/3′-BHQ-3) or similar FRET pairs available from Biosearch Technologies, Inc. of Novato, Calif., fluorescent quantum dots (stable long lived fluorescent donors), tags with nmr active groups, Raman active tags, tags with spectral features that can be easily identified such as IR, far IR, near IR, visible UV, far UV or the like. It should be recognized that any molecule, nano-structure, or other chemical structure that is capable of chemical modification and includes a detectable property capable of being detected by a detection system. Such detectable structure can include one presently known and structures that are being currently designed and those that will be prepared in the future.

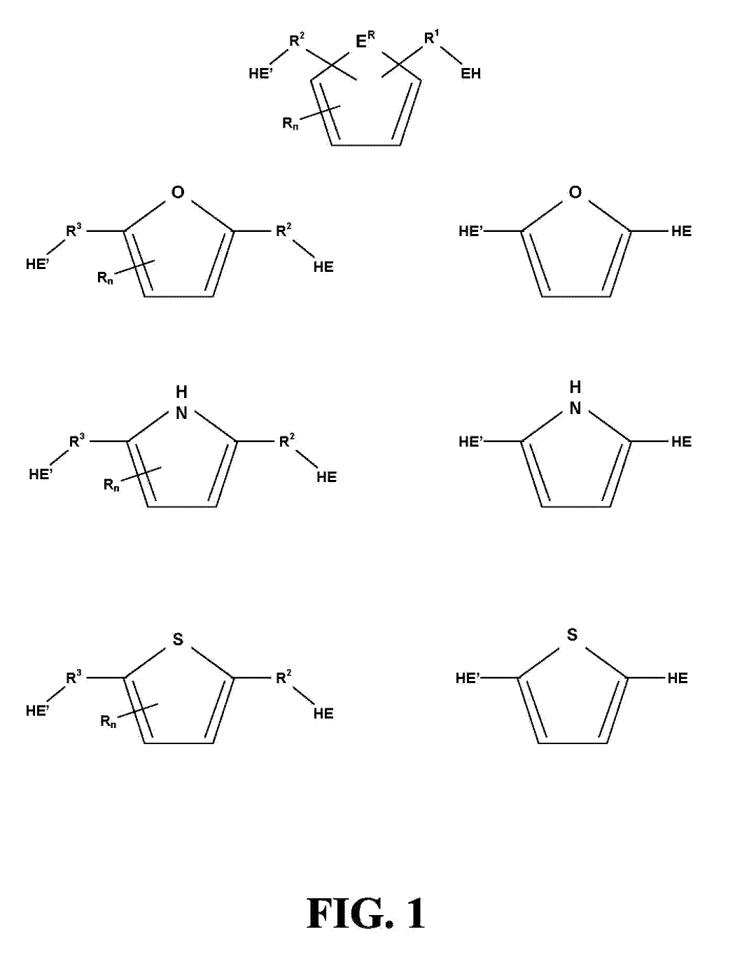

Referring now to FIG. 1, a set of exemplary single ring linkers are shown, where E^{R} is a main element containing group such as CH, SiH, N, P, or the like. The ring structure can also be saturated or unsaturated, but not aromatic in which case E^{R} is a main element containing group such as CH, SiH, N, P, O, S, or the like. R is a carbyl group and n is an integer having a value between 1 and the maximum number of R groups that the ring structure can accommodate and still be a compound known or capable of synthesis by known synthetic methods.

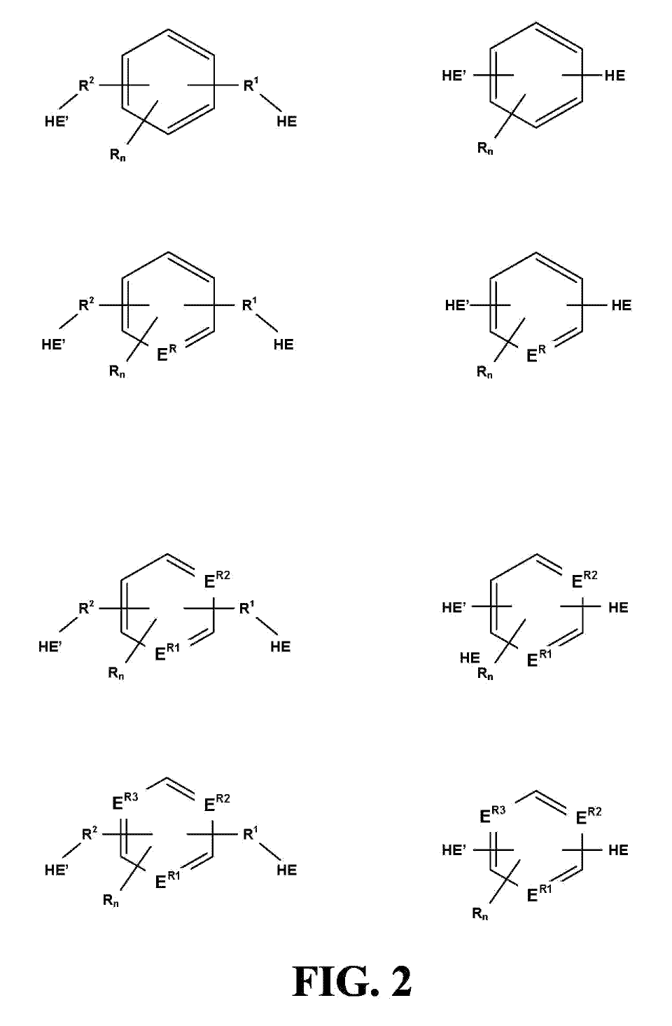

Referring now to FIG. 2, a set of exemplary single ring linkers are shown, where E^{R1}, E^{R2} and E^{R3} are the same or different main element containing groups such as CH, SiH, N, P, or the like. The ring structure can also be saturated or unsaturated, but not aromatic in which case E^{R1}, E^{R2} and E^{R3} are the same or different main element containing groups such as CH, SiH, N, P, O, S, or the like. R is a carbyl group and n is an integer having a value between 1 and the maximum number of R groups that the ring structure can accommodate and still be a compound known or capable of synthesis by known synthetic methods.

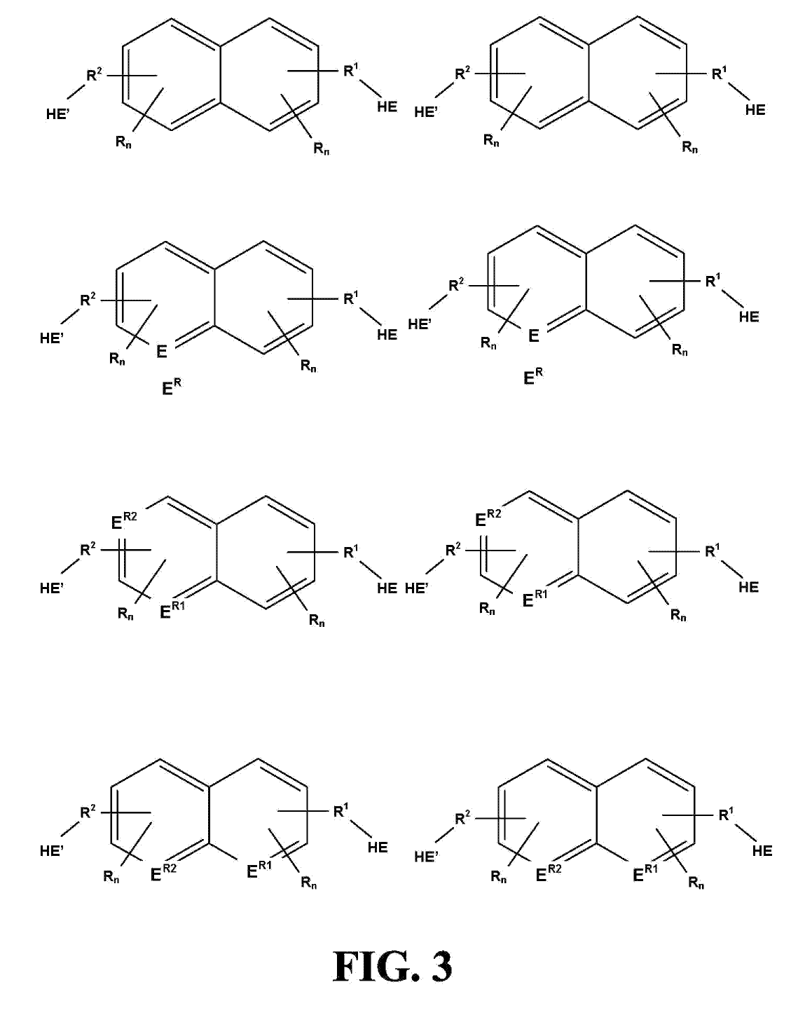

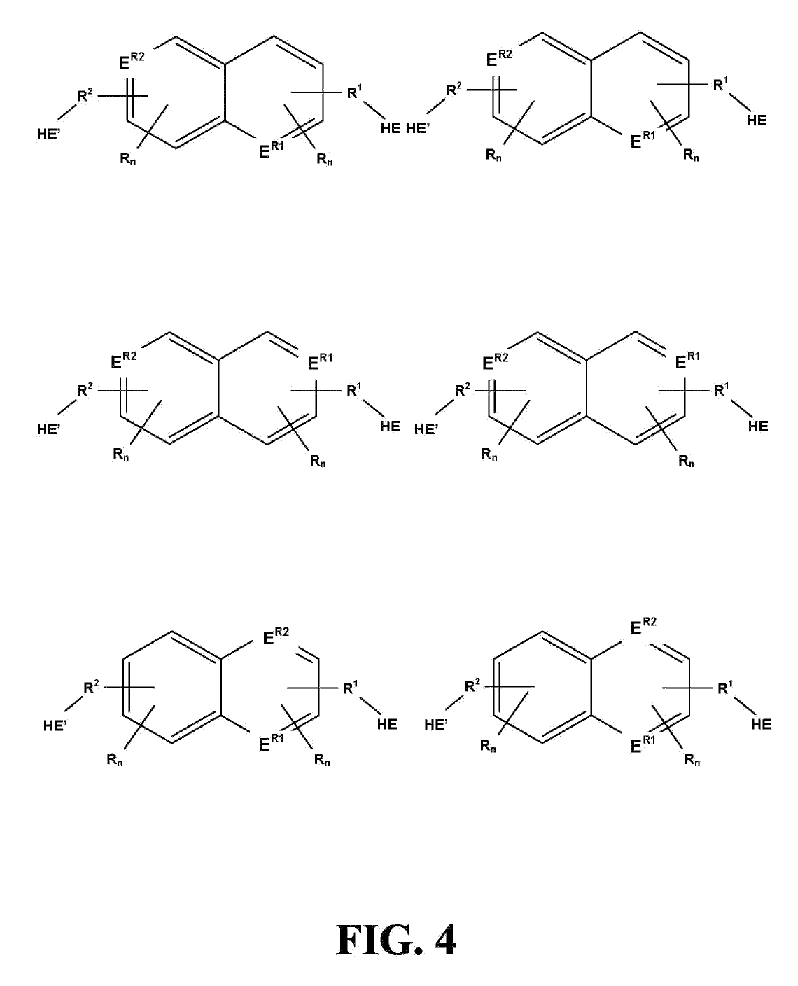

Referring now to FIGS. 3&4, a set of exemplary binary ring linkers are shown, where E^{R1} and E^{R2} are the same or different main element containing groups such as CH, SiH, N, P, or the like. The ring structure can also be saturated or unsaturated, but not aromatic in which case E^{R1} and E^{R2} are the same or different main element containing groups such as CH, SiH, N, P, O, S, or the like. R is a carbyl group and n is an integer having a value between 1 and the maximum number of R groups that the ring structure can accommodate and still be a compound known or capable of synthesis by known synthetic methods. It should be recognized that the second ring can be any other sized ring besides a six membered ring.

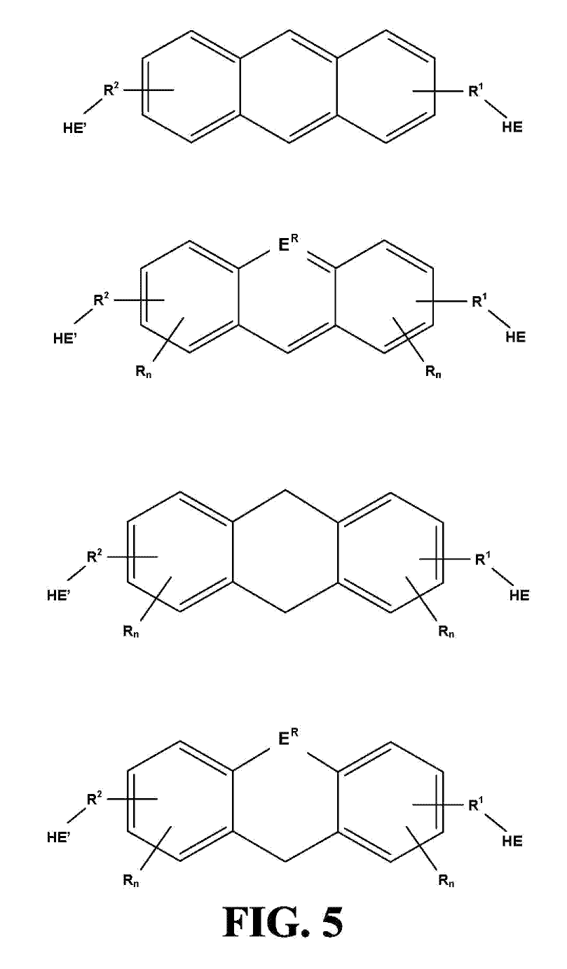

Referring now to FIG. 5, a set of exemplary trinary ring linkers are shown, where E^{R} is a main element containing group such as CH, SiH, N, P, or the like. The ring structure can also be saturated or unsaturated, but not aromatic in which case E^{R} is a main element containing group such as CH, SiH, N, P, O, S, or the like. R is a carbyl group and n is an integer having a value between 1 and the maximum number of R groups that the ring structure can accommodate and still be a compound known or capable of synthesis by known synthetic methods. It should be recognized that the second ring can be any other sized ring besides a six membered ring.

### EXPERIMENTS OF THE INVENTION

### Example 1

This example illustrates the preparation of dATP bonded to 1,4-Bis-(3-aminopropyl)piperazine to form dATP-BAPP.

20 nmol of the sodium salt of dATP was treated with Dowex resin/TEAB, lyophilized and dried under vacuum. Dry DCC (75 μmol) was added to a DMF (200 μL) solution of the above nucleotide and the resulting mixture was stirred under argon for 2 hrs. Pyridine (17 μL) was added and the resulting mixture was slowly evaporated. To the pellet was added a solution of 1,4-Bis-(3-aminopropyl)piperazine (150 μmol) in DMF (200 μL) and the solution was stirred for 12 hrs. The mixture was then quenched with water and centrifuged to remove the solid. The clear solution was subject to HPLC (SAX, TEAB) purification. The product was collected and lyophilized. The pellet was dissolved in HEPES buffer (10 mM, pH 8.5). Yield 4.2 μmol, 21%. Note: The yield is not accurate because aliquots from the DCC-reacted mixture were transferred into several reactions including the top one. Normally, the yield of this synthesis is much higher (>50%).

### Example 2

This example illustrates the preparation of the compound of Example 1 bonded to ROX to form dATP-BAPP-ROX.

The above nucleotide dATP-11 (0.5 μmol) was reacted with ROX-SE (2 μmol) overnight in the following mixture: DMF (20 μL)+NaHCO₃ (1 M, pH 9). The product was purified on a Sephadex G25 column and then on HPLC (C18, TEAA/MeOH). Yield 0.41 μmol, 82%.

### Example 3

This example illustrates an enzymatic tests on dATP-BAPP-ROX.

This nucleotide was tested upon calf intestinal alkaline phosphatese (CIAP) and phosphodiesterase 1 (PDE1) and the result was analyzed on PEI cellulose thin-layer chromatography. It was inert to CIAP and readily hydrolyzed by PDE 1.

Fluorophores

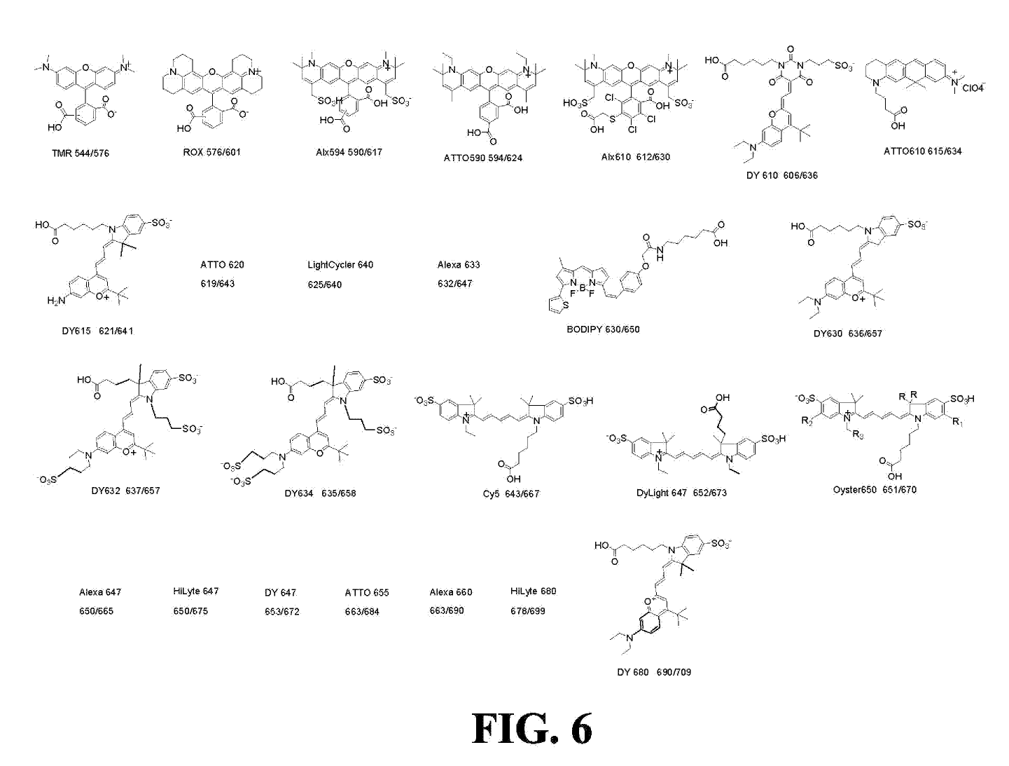

Referring now to FIG. 6, fluorophores used in the synthesis of fluorophore modified dNTPs are shown.

Pictorial Examples of dNTP Modification Schemes

Referring now to FIG. 7-11, a number of synthetic schemes for prepare modified dNTPs are shown.

I. General Synthetic Scheme for Nitrogen Terminated Linkers

This general scheme involves coupling an dNTP to a nitrogen-terminated linker in the presence of N-ethyl-N′-(3-dimethylaminopropyl)carbodiimide (EDC) in two steps.

Step 1—dNTP-1(TEA⁺)

Nucleotide dNTP Na₂ (12.7 μmol) is reacted with linker 1 (110 μmol) in the presence of EDC (110 μmol) at rt for 3 hr and pH is maintained at ˜5.7 over the time. The product is purified on HPLC (C18) with TEAA/MeOH or on HPLC (SAX) with TEAB. The product after lyophilization is dissolved in HEPES buffer (10 mM, pH 8.5). Yield varies from 35% to 55%.

Step 1—dNTP-1-dye (TEA⁺)

The intermediate dNTP-1 (1 μmol) in NaHCO₃ buffer (1M, pH9, 50 μL) and dye-NHS (2.5 μmol) in DMF (100 μL) are mixed and reacted overnight. After a Sephadex G-25 column purification, the product-containing sample is purified on HPLC (C18) with TEAA/MeOH or on HPLC (SAX) with TEAB. The product after lyophilization is dissolved in HEPES buffer (10 mM, pH8.5). Yield varies from 15% to 70%. Enzymatic assay and MS are performed when necessary.

II. Alternate General Synthetic Scheme for Nitrogen-Terminated Linkers

This general scheme involves activation of dNTP by DCC and then coupling the intermediate to a linker.

Step 1—dNTP(TEA⁺)

Nucleotide dNTP Na₂ (57 μmol) is passed through a TEAB-equilibrated Dowex resin (H⁺) column. The sample is lyophilized.

Step 2—dNTP-2 (TEA⁺)

Nucleotide dNTP TEA (20 nmol) is coevaporated with TEA and methanol 3 times before dried under vacuum overnight. DCC (75 nmol) is dried under vacuum 2 hrs. Linker compound (200 μmol) is coevaporated with TEA and methanol and dried under vacuum overnight.

DCC is transferred to dNTP TEA in DMF/MeOH (200 μL/20 μL) and the mixture is stirred at r.t. for 3-4 hrs before coevaporated with pyridine (17 μL). Linker compound in DMF (200-300 μL) is then added to the pellet and the resulting solution is stirred at r.t. overnight. The product is purified on HPLC (SAX column, TEAB). After lyophilization the product is dissolved in HEPES buffer (10 mM, pH 8.5). Yield varies from 30% to 50%.

Step 3—dNTP-2-dye (TEA⁺)

This step is similar to Step 2 of the first general scheme.

III. General Synthetic Scheme for Linkers Terminated by Nitrogen at One End and Oxygen at the Other End

This scheme involves activating a monophosphate with CDI and coupling the intermediate to a dNDP.

Step 1—dNDP (TBA+)

Nucleotide dNDP sodium salt (43 umol) is passed through Dowex resin (H+) into cooled TBA. It is coevaporated with DMF 3 times and dried under vacuum overnight.

Step 2-Pi-5-Cbz (TBA+)

Alcohol 5-Cbz is phosphorylated with POCl₃/P(OMe)₃ system and purified on Sephadex G25 DEAE anion exchanger with a gradient of AB buffer. After lyophilization it was transformed into TBA+ salt as described in Step 1. Yield varies from 50% to 70%.

Step 3—dNTP-5-Cbz (TEA+)

All reagents are dried under vacuum. Monophosphate Pi-5-Cbz (TBA+, 33 μmol)) is treated with CDI (165 umol) in DMF (250 μL) for 6 hrs before MeOH (264 umol) is added to quench the excess of CDI. Nucleotide dNDP (TBA+, 43 umol) is added in DMF (400 μL) and the reaction is allowed overnight. Purification was achieved on HPLC (SAX, TEAB) followed by lyophilization. Yields vary from 20% to 50%.

Step 4—dNTP-5 (TEA⁺)

Nucleotide dNTP-5-Cbz (TEA+) is treated with ammonium formate and Pd/C for 10-20 minutes. The product is purified on HPLC (SAX, TEAB) followed by lyophilization. Yields are above 90%.

Step 5—dNTP-5-dye (TEA⁺)

Same procedure as performed for the second step of the first general procedure.

Overview

1. dNDP (1) is converted to tetrabutylammonium salt (2) by cation exchange. This converts the diphosphate into a reagent that is very soluble in dry organic solvents
2. Potential hydroxyl terminated linker (3) (containing a nitrogen protecting group, as example here, trifluoroactetate (TFA)) is phosphorylated with phosphorous oxytrichloride to yield activated chlorophosphate (4). Excess POCl₃ is removed by evaporation yielding the dichloride ester
3. (2) and (4) are reacted in an anhydrous solvent (for example dry DMF), followed by hydrolysis of the resulting (monochloro)triphosphate ester to the triphosphate (5)
   Procedure for Preparing the dNTP with Linker Attached to γ-Phosphate Through P—O Linkage

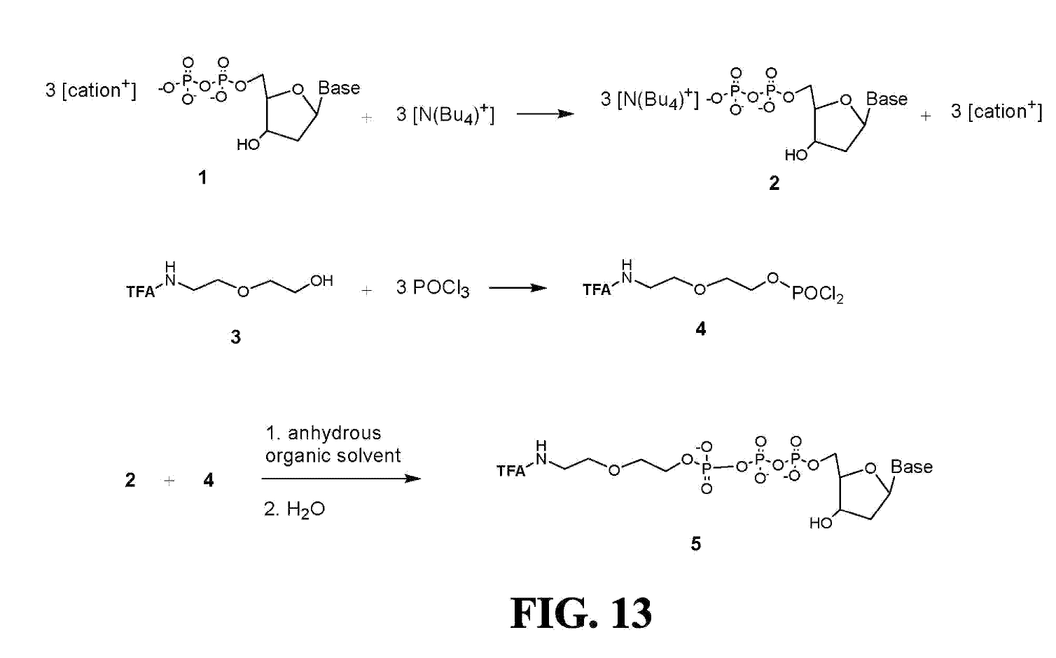

Step 1—See FIG. 13.

This step illustrates the conversion of a dNDP-sodium salt (1) to a dNDP-tetrabutylammonium salt (2).

An aqueous solution (2 mL) of the commercially available dNDP-sodium salt (100 to 150 mg) was loaded onto a strong cation exchange (—SO₃H) packed column. The column was eluted with gravity. Fractions were collected and checked by spotting on TLC and visualized by UV lamp (dNDP will have show blue spot under UV). The desired fractions were pooled together and quenched with tetrabutylammonium hydroxide (1.01 eq in˜10 mL H₂O) immediately at 0° C. The solution was evaporated to dryness. The residue was re-dissolved in DMF and dried down. When this material was dried down 3× with DMF, the dNDP-Tetrabutylammonium salt was ready for the coupling reaction.

Step 2

This step illustrates the coupling of the linker (3) to dNDP (2) using dCDP and neutral EO linker as an example.

Material

TFA protected linker (3): 10 mg (0.0497 mmole; dried by co-evaporating with DMF three times before use); POCL₃: 9.2 mL (0.0994 mmole, 2×); dCDP-tetrabutylammonium salt (2) (24.85 μmole, quantity determined by UV absorbance at 260 nm with ε˜9,300); dry methylene dichloride (DCM); dry dimethylformamide (DMF); 1.5 M triethylammonium bicarbonate (TEAB) buffer (pH ˜7.5 to 8).

A solution of the linker (3) in dry DCM (0.5 mL) was added into the solution of POCl₃ in DCM (1 mL), at 0° C. The reaction was then stirred at 0° C. for three hours. The solution was then evaporated to dryness under reduced pressure and was further dried down with high vacuum for another 10 minutes to remove the residual POCl₃. The residue (4) was then re-dissolved in dry DMF (1 mL). To this solution, dCDP (2) (in dry 0.5 mL of dry DMF) was added in at 0° C. The reaction was then stirred at 0° C. initially and then the temperature was gradually raised to ambient. The reaction was then stirred at room temperature overnight, followed by quenching with the addition of TEAB buffer (5 mL) at 0° C. The mixture was then stirred at 0° C. for three more hours. The product (5) was evaporated to dryness, re-dissolved in water, material and purified by reverse phase HPLC (C-18 column).

The linkers tabulated in Table 1 were used in the above articulated preparatory methods.

TABLE 1
Linker Used in the Various Preparatory Methods

| Linker | Structure |
|---|---|
| 1 | 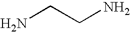 |
| 2 | 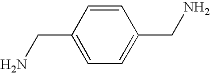 |
| 3 | 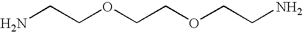 |
| 4 | 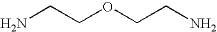 |
| 5 | 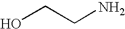 |
| 6 | 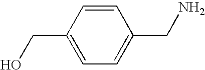 |
| 7 | 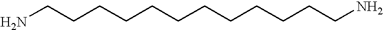 |
| 8 | 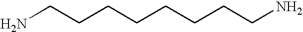 |
| 9 | 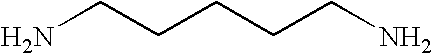 |
| 10 | 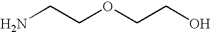 |
| 11 | 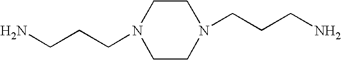 |
| 12 | 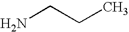 |

The modified nucleotides tabulated in Table 2 were prepared using the above articulated preparatory methods. In Table 2, the numbers represent the linkers tabulated above. The structures with Cbz are protected linker structures where the terminal amino group has been reacted with benzoic acid.

TABLE 2
Modified Nucleotides Prepare Using
the Various Preparatory Methods
Modified Nucleotide

ATP-1
ATP-1-ATTO620
ATP-1-Biotin
ATP-1-Cy3
ATP-1-Cy5
ATP-1-Fluorescein
ATP-1-Fluoresecein1
ATP-1-Fluoresecein2
ATP-1-ROX
ATP-1-ROX (Na+)
ATP-1-ROX(S)
ATP-1-ROX(6)
ATP-1-TMR1
ATP-1-TMR2
ATP-2
ATP-2-biotin
ATP-2-Cy3
ATP-2-Cy5
ATP-2-Flu
ATP-2-ROX
ATP-2-ROX(5)
ATP-2-ROX(6)
ATP-2-TMR-1
ATP-2-TMR-2
ATP-3
ATP-3-biotin-1
ATP-3-Cy3
ATP-3-Cy5
ATP-3-Flu
ATP-3-ROX
ATP-3-TMR
ATP-4
ATP-4-Biotin
ATP-4-Cy3
ATP-4-Cy5
ATP-4-Flu
ATP-4-ROX
ATP-4-TMR
ATP-5
ATP-5-Cbz
ATP-5-ROX
ATP-6
ATP-6-Cbz
ATP-7
ATP-8
ATP-9
ATP-P5-Cbz
ATPP-5
ATPP-5-biotin
ATPP-5Cbz
ATPP-5ROX
dATP-1
dATP-1-BODIPY
dATP-1-Cy5
dATP-1-LC
dATP-1-ROX
dATP-2
dATP-2-Alx594
dATP-2-Alx610
dATP-2-Alx633
dATP-2-BODIPY
dATP-2-Cy5
dATP-2-Oys650
dATP-2-ROX
dATP-3
dATP-3-Bodipy
dATP-3-Cy5
dATP-3-ROX
dATP-4
dATP-4-Cy5
dATP-4-ROX
dATP-5
dATP-5-Cbz
dATPP-5-Cbz
dATPP-5
dATPP-5-Cbz
dATPP-5-ROX
dATPP-5-ROX
dATP-6
dATP-6-Cbz
dATP-6-ROX-1
dATP-6-ROX-2
dATP-10
dATP-10-Cbz
dATP-10-ROX
dATP-11
dATP-11-ROX
dATP-12
dATP-12-Cbz
dATP-12-ROX
dCTP-1
dCTP-1-Cy5
dCTP-1-LC
dCTP-1-ROX
dCTP-1-TMR
dCTP-1-TMR1
dCTP-1-TMR2
dCTP-2
dCTP-2
dCTP-2-Alx610
dCTP-2-Alx633
dCTP-2-Cy5
dCTP-2-ROX
dCTP-3
dCTP-3-Cy5
dCTP-3-ROX
dCTP-4
dCTP-4-Cy5
dCTP-4-ROX
dCTP-6
dCTP-6-Alx610
dGTP-1
dGTP-1-ATTO620
dGTP-1-Cy5
dGTP-1-LC
dGTP-1-LC
dGTP-1-ROX
dGTP-2
dGTP-2-Alx594
dGTP-2-Alx610
dGTP-2-Alx633
dGTP-2-Cy5
dGTP-2-Oyster650
dGTP-2-ROX
dGTP-2-ROX
dGTP-3
dGTP-3-Cy5
dGTP-3-ROX
dGTP-4
dGTP-4-Cy5
dGTP-4-ROX
dTTP-1
dTTP-1-Cy5
dTTP-1-LC
dTTP-1-ROX
dTTP-1-TMR
dTTP-1-TMR1
dTTP-1-TMR2
dTTP-2
dTTP-2-Alx610
dTTP-2-Alx633
dTTP-2-Cy5
dTTP-2-ROX
dTTP-3
dTTP-3-Cy5
dTTP-3-ROX
dTTP-4
dTTP-4-Cy5
dTTP-4-ROX
dTTP-5
dTTP-5-Cbz
dTTP-5-Cbz
dTTP-5-ROX
dUTP-1
dUTP-1-Cy5
dUTP-1-LC
dUTP-1-LC1
dUTP-1-LC2
dUTP-1-ROX
dUTP-1-TMR1
dUTP-1-TMR2

Linker 10—1,1′-Carbonyldiimidazole (CDI) Chemistry

Pi-10-Cbz (Bu₃NH⁺) was vigorously dried down to get the weight for quantification. Its coupling with dADP (20 μmol scale) was tried again with new reagents and still ended with a low yield (TLC & HPLC). The product, however, was purified on HPLC (SAX, TEAB) to give 0.53 μmol of dATP-10-Cbz. After hydrogenolysis deprotection (0.4 umol scale), an aliquot of dATP-10 (44 nmol) was directly labeled with ROX-SE. After Sephadex G-25 column and C18 HPLC purification, dATP-10-ROX (3.8 nmol) was submitted to enzymology team to evaluate its polymerase incorporation.

Linker 10—Trifluoroacetic Anhydride/Methylimidazole Chemistry

The method reacts monophosphate with trifluoroacetic anhydride and the resulting mixed anhydride is reacted with methylimidazole followed by dADP quenching. It was tested at 20 μmol scale for the preparation of dATP-10-Cbz and gave a low yield of 0.5 μmol. Although not widely used this chemistry takes advantage of the volatility of (CF₃CO)₂O and CF₃COOH and represents a fast coupling method (<3 hrs) compared to other known methods.

All references cited herein are incorporated by reference. Although the invention has been disclosed with reference to its embodiments, from reading this description those of skill in the art may appreciate changes and modification that may be made which do not depart from the scope and spirit of the invention as described above and claimed hereafter.

We claim:

1. A modified nucleotide of the general formula (I):
DG-E′-G-E-Nu (I)
where:
DG is a detectable group,
E and E′ are the same and different group,
G is a linking group, and
Nu is a natural or synthetic nucleotide,
where G comprises a linear or branched alkenyl group or an alkenyl group including a central ring structure.

2. The nucleotide of claim 1, wherein the E and E′ group include a central main group element.

3. The nucleotide of claim 1, wherein the central main group elements are selected from the group consisting of boron (B), carbon (C), nitrogen (N), oxygen (O), silicon (Si), phosphorus (P), sulfur (S), gallium (Ga) and germanium (Ge).

4. The nucleotide of claim 1, wherein G includes a central ring structure.

5. The nucleotide of claim 1, wherein G includes a linear alkenyl group.

6. The nucleotide of claim 1, wherein the nucleotide has the general formula (II):
DG-E′-R²-A—R¹-E-Nu (II)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O). (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure,
R¹ and R² are the same or different and are carbenzyl groups,
A is a ring structure, and
Nu is a natural or synthetic nucleotide.

7. The nucleotide of claim 1, wherein the nucleotide has the general formulas (III or IIIa):
DG-E′-R²-A-R¹-E-P(O₂)OP(O₂)OP(O₂)-Sugar-Base (III)
DG-E′-R²-A-R¹-E-P(O₂)OP(OZ¹)OP(OZ²)-Sugar-Base (IIIa)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure,
R¹ and R² are the same or different and are is carbenzyl groups,
A is a ring structure, Sugar is a sugar moiety,
Base is a natural or synthetic nucleotide base and
Z¹ or Z² are the same or different and are groups that either modify incorporation timing or enhancing detection of the detectable group.

8. The nucleotide of claim 1, wherein the nucleotide has the general formulas (IV or IVa):
DG-E′-R²-A-R¹-E-P(O)(OP(O₂)OH)OP(O₂)-Sugar-Base (IV)
DG-E′-R²-A-R¹-E-P(O)(OP(OZ¹)OH)OP(OZ²)-Sugar-Base (IVa)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O). (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure,
R¹ and R² are the same or different and are is carbenzyl groups,
A is a ring structure,
Sugar is a sugar moiety,
Base is a natural or synthetic nucleotide base and
Z¹ or Z² are the same or different and are groups that either modify incorporation timing or enhancing detection of the detectable group.

9. The nucleotide of claim 1, wherein the nucleotide has the general formula (V):
DG-E′-R²-A-R¹-E-P(O)(OP(O₂)OP(O₂)OH)-Sugar-Base (V)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure,
R¹ and R² are the same or different and are is carbenzyl groups,
A is a ring structure,
Sugar is a sugar moiety, and
Base is a natural or synthetic nucleotide base.

10. The nucleotide of claim 1, wherein the nucleotide has the general formula (VI):
DG-E′-R²-A-R¹-E-Sugar(P(O₂)OP(O₂)OP(O₂)OH)Base (VI)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure,
R¹ and R² are the same or different and are is carbenzyl groups,
A is a ring structure,
Sugar is a sugar moiety, and
Base is a natural or synthetic nucleotide base.

11. The nucleotide of claim 1, wherein the nucleotide has the general formula (VII):
DG-E′-R²-A-R¹-E-Base-Sugar-P(O₂)OP(O₂)OP(O₂)OH (VII)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure,
R¹ and R² are the same or different and are is carbenzyl groups,
A is a ring structure,
Sugar is a sugar moiety, and
Base is a natural or synthetic nucleotide base.

12. The nucleotide of claim 1, wherein the ring structure A is saturated, unsaturated or aromatic or can include a mixture of saturated, unsaturated, or aromatic rings.

13. The nucleotide of claim 1, wherein the each ring in the ring structure includes from to about 12 main group elements.

14. The nucleotide of claim 1, wherein each carbyl group and each carbenzyl group include from 1 to 40 carbon, where one or more of the carbon atoms can be replaced with a hetero atoms selected from the group consisting of B, C, Si, Ge, N, P. As, O, S, or Se and having sufficient hydrogen atoms to satisfy the valency of the group, where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions.

15. The nucleotide of claim 1, wherein the nucleotide has the general formula (VIII):
DG-E′-R-E-Nu (VIII)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group, and
Nu is a natural or synthetic nucleotide.

16. The nucleotide of claim 1, wherein the nucleotide has the general formulas (IX or IXa):
DG-E′-R-E-P(O₂)OP(O₂)OP(O₂)-Sugar-Base (IX)
DG-E′-R-E-P(O₂)OP(OZ¹)OP(OZ²)-Sugar-Base (IXa)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety,
Base is a natural or synthetic nucleotide base, and
Z¹ or Z² are the same or different and are groups that either modify incorporation timing or enhancing detection of the detectable group as described herein.

17. The nucleotide of claim 1, wherein the nucleotide has the general formulas (X or Xa):
DG-E′-R-E-P(O)(OP(O₂)OH)OP(O₂)-Sugar-Base (X)
DG-E′-R-E-P(O)(OP(OZ¹)OH)OP(OZ²)-Sugar-Base (Xa)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety,
Base is a natural or synthetic nucleotide base, and
Z¹ or Z² are the same or different and are groups that either modify incorporation timing or enhancing detection of the detectable group as described herein.

18. The nucleotide of claim 1, wherein the nucleotide has the general formula (XI):
DG-E′-R-E-P(O)(OP(O₂)OP(O₂)OH)-Sugar-Base (XI)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety, and
Base is a natural or synthetic nucleotide base.

19. The nucleotide of claim 1, wherein the nucleotide has the general formula (XII):
DG-E′-R-E-Sugar(P(O₂)OP(O₂)OP(O₂)OH)Base (XII)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety, and
Base is a natural or synthetic nucleotide base.

20. The nucleotide of claim 1, wherein the nucleotide has the general formula (XIII):
DG-E′-R-E-Base-Sugar-P(O₂)OP(O₂)OP(O₂)OH (XIII)
where:
DG is a detectable group,
E and E′ are the same and different and are a carbon group (C(H)₂, C(HR³) or C(R³)₂), an oxygen atom (O), a sulfur atom (S), an amino group (N(R³)), an phosphano group (P(R³)), a phosphito group (P(OR³)O), a phosphate group (P(O₂)O), a polyphosphate group (P(O₂)O)ₙ (n is an integer having a value between 3 and 12), a silyl group (Si(R³)₂), a siloxyl group (Si(OR³)₂), a carboxy group (C(O)O), a keto group (C(O)), an amido group (C(O)N(R³)), an urea group (N(R³)C(O)N(R³)), a carbonate group (OC(O)O), or an urethane group (OC(O)N(R³), R³ is a hydrogen atom, a carbyl group or is absent depending on the structure (e.g., E′ or E is a nitrogen atom doubly bonded to DG or to R² or a carbon atom triply bonded to DG or R²),
R is a carbenzyl group,
Sugar is a sugar moiety, and
Base is a natural or synthetic nucleotide base.

21. The nucleotide of claim 1, wherein the each carbyl group and each carbenzyl group include from 1 to 40 carbon, where one or more of the carbon atoms can be replaced with a hetero atoms selected from the group consisting of B, C, Si, Ge, N, P. As, O, S, or Se and having sufficient hydrogen atoms to satisfy the valency of the group, where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions.

22. A method for preparing gamma phosphate modified nucleotide triphosphates comprising the steps of:
cyclizing a nucleotide triphosphate in N,N-dicyclohexylcarbodiimide (DCC) to form a cyclized nucleotide triphosphate,
contacting the cyclized nucleotide triphosphate with an α,ω-diamino linker, where the linker includes a linking group comprising a linear or branched carbenzyl group or a carbenzyl group including a central ring structure, to form a linker gamma phosphate modified nucleotide triphosphate, and
contacting the linker gamma phosphate modified nucleotide triphosphate with a carboxylc acid, a carboxylic acid chloride or a carboxylic acid anhydride including a detectable group having a detectable property to form a detectable group, linker gamma phosphate modified nucleotide triphosphate.

23. A method for preparing gamma phosphate modified nucleotide triphosphates comprising the steps of:
contacting a nucleotide triphosphate with an α,ω-diamino linker, where the linker includes a linking group comprising a linear or branched carbenzyl group or a carbenzyl group including a central ring structure, in N-ethyl-N′-(3-dimethylaminopropyl)carbodiimide (EDC) to form a linker gamma phosphate modified nucleotide triphosphate, and contacting the linker gamma phosphate modified nucleotide triphosphate with a carboxyic
acid, a carboxylic acid chloride or a carboxylic acid anhydride including a detectable group having a detectable property to form a detectable group, linker gamma phosphate modified nucleotide triphosphate.

24. A method for preparing gamma phosphate modified nucleotide triphosphates comprising the steps of:
contacting a protected linker of the general formula Q-NH-G-OH with a phosphate donor to form a phosphate terminated linker of the general formula Q-NH-G-OP(O)(OH)₂,
where Q is a protecting group and G a linker comprising a linear or branched carbenzyl group or a carbenzyl group including a central ring structure,
contacting the phosphate terminated linker, Q-NH-G-OP(O)(OH)₂, with imidazole to form an imidazole activated phosphate terminated linker, Q-NH-G-OP(O)(OH)-imidazole, contacting the imidazole activated phosphate terminated linker, Q-NH-G-OP(O)(OH)-imidazole with a nucleotide polyphosphate to form a protected, linker terminal phosphate modified nucleotide polyphosphate, Q-NH-G-O—[P(O)(OH)]ₙ—O-Nuc, where n is an integer having a value between 3 and 12,
deprotecting the protected gamma phosphate functionalized nucleotide polyphosphate, Q NH-G-O—[P(O)(OH)]ₙ—O-Nuc, to form an unprotected linker, gamma phosphate modified nucleotide polyphosphate, H₂N-G-O—[P(O)(OH)]ₙ—O-Nuc, and
contacting the unprotected linker, gamma phosphate modified nucleotide polyphosphate, H₂N-G-O—[P(O)(OH)]ₙ—-Nuc, with a carboxylc acid, a carboxylic acid chloride or a carboxylic acid anhydride including a detectable group (DG) having a detectable property to form a detectable group gamma phosphate modified nucleotide triphosphate, DG-HN-G-O—[P(O)(OH)]ₙ—O-Nuc.

25. A method for preparing gamma phosphate modified nucleotide triphosphates comprising the steps of:
contacting a linker of the general formula Q-NH-G-OH with a sulfonate donor to form a sulfonate terminated linker, Q-NH-G-OS(O)₂CH₃,
contacting the sulfonate terminated linker, Q-NH-G-OS(O)₂CH₃, with phosphate donor to form a phosphate terminated linker, Q-NH-G-OP(O)(OH)₂,
contacting the phosphate terminated linker, Q-NH-G-OP(O)(OH)₂, with imidazole to form an activated phosphate terminated linker, Q-NH-G-OP(O)(OH)-Imidazole, contacting the imidazole activated phosphate terminated linker, Q-NH-G-OP(O)(OH)-Imidazole, with a nucleotide polyphosphate to form a protected linker, terminal phosphate modified nucleotide polyphosphate, Q-NH-G-O—[P(O)(OH)]ₙ—-Nuc, where n is an integer having a value between 3 and 12,
deprotecting the protected gamma phosphate functionalized nucleotide polyphosphate, Q-NH-G-O—[P(O)(OH)]ₙ—O-Nuc, to form an unprotected linker, terminal phosphate modified nucleotide polyphosphate, H₂N-G-O—[P(O)(OH)]ₙ—O-Nuc, and
contacting the unprotected linker, terminal phosphate modified nucleotide polyphosphate, H₂N-G-O—[P(O)(OH)]ₙ—O-Nuc, with a carboxylc acid, a carboxylic acid chloride or a carboxylic acid anhydride including a detectable group having a detectable property to form a detectable group, linker, terminal phosphate modified nucleotide triphosphate, DG-HN-G-O—[P(O)(OH)]ₙ—O-Nuc.

26. A method for preparing gamma phosphate modified nucleotide triphosphates comprising the steps of:
contacting a linker of the general formula H₂N-G-OH with trifluoro acetic acid (TFA) to form a TFA terminated linker, TFA-NH-G-OH,
contacting the TFA terminated linker, TFA-NH-G-OH, with a cyclized nucleotide triphosphate in the presence of a base to form a TFA terminated linker, gamma phosphate modified nucleotide triphosphate, TFA-NH-G-[P(O)(OH)]₃—O-Nuc,
deprotecting the TFA terminated linker, gamma phosphate modified nucleotide triphosphate, TFA-NH-G-[P(O)(OH)]₃—O-Nuc, to form a linker, gamma phosphate modified nucleotide triphosphate, H₂N-G-[P(O)(OH)]₃—O-Nuc, and
contacting the a linker, gamma phosphate modified nucleotide triphosphate, H₂N-G-[P(O)(OH)]₃—O-Nuc, with a carboxylc acid, a carboxylic acid chloride or a carboxylic acid anhydride including a detectable group having a detectable property to form a detectable group, linker, terminal phosphate modified nucleotide triphosphate, DG-HN-G-O—[P(O)(OH)]₃—O-Nuc.

27. A method for preparing gamma phosphate modified nucleotide triphosphates comprising the steps of:
contacting a nucleotide diphosphate salt, Nuc-O—P(O)(O⁻)—O—P(O)(O⁻)₂M₃, with a tetracarbyl ammonium salt, R₄N⁺X⁻, to form a nucleotide diphosphate tetracarbyl ammonium salt, Nuc-O—P(O)(O⁻)—O—P(O)(O⁻)₂(R₄N⁺)₃,
contacting an N-TFA-protected, α-amino, ω-hydroxy linker, TFA-N(H)-G-OH, with sufficient POCl₃ to form an N-TFA-protected, α-amino, ω-dichlorophosphite linker, TFA-N(H)-G-OP(O)Cl₂,
contacting the nucleotide diphosphate tetracarbyl ammonium salt, Nuc-O—P(O)(O⁻)—O—P(O)(O⁻)₂(R₄N⁺)₃, with the N-TFA-protected, α-amino, ω-dichlorophosphite linker, TFA-N(H)-G-OP(O)Cl₂, to form a TFA-protected, α-amino, ω-gamma phosphate modified nucleotide triphosphate tetracarbyl ammonium salt, TFA-NH-G-O—[P(O)(O⁻)]₃—O-Nuc((R₄N⁺)₃,
deprotecting the TFA-protected, α-amino, ω-gamma phosphate modified nucleotide triphosphate tetracarbyl ammonium salt, TFA-NH-G-O—[P(O)(O⁻)]₃—O-Nuc((R₄N⁺)₃, to form a linker, gamma phosphate modified nucleotide triphosphate, α-amino, ω-gamma phosphate modified nucleotide triphosphate tetracarbyl ammonium salt, H₂N-G-O—[P(O)(O⁻)]₃—O-Nuc((R₄N⁺)₃.
